(19) United States
(12) Patent Application Publication
(10) Pub. No.: US 2026/0260733 A1
(43) Pub. Date: Sep. 3, 2026

(54) IMAGE PROCESSING DEVICE, IMAGE PROCESSING METHOD, AND STORAGE MEDIUM

(71) Applicant: NEC Corporation, Tokyo (JP)

(72) Inventors: Terumi UMEMATSU, Tokyo (JP); Yoshifumi ONISHI, Tokyo (JP)

(73) Assignee: NEC Corporation, Tokyo (JP)

(21) Appl. No.: 19/164,730

(22) PCT Filed: Mar. 27, 2023
(86) PCT No.: PCT/JP2023/012154

§ 371 Date: Sep. 12, 2025

(51) Int. Cl. G16H 30/40; A61B 3/11; A61B 3/14; G16H 20/10; H04N 23/60

(52) U.S. Cl. CPC G16H 30/40; A61B 3/112; A61B 3/14; G16H 20/10; H04N 23/64

(57) ABSTRACT

The image processing device mainly includes a medication information acquisition means 14X, a photographing timing determination means 15X, and a pupil information generation means 17X. The medication information acquisition means 14X acquires medication information on a medicine used by a subject. The photographing timing determination means 15X determines a photographing timing of the subject by a photographing means based on the medication information. The pupil information generation means 17X generates pupil information relating to a pupil of the subject based on an image generated by the photographing means at the photographing timing.

### TECHNICAL FIELD

The present disclosure relates to a technical field of an image processing device, an image processing method, and a storage medium that perform processing relating to determination of a medication effect by an image.

### BACKGROUND ART

A system that measures a pupil of a subject based on a face image obtained by photographing the subject is known. For example, PTL 1 discloses a system that detects a region of a pupil of a subject based on video signals from two cameras and analyzes a pupil area, a pupil diameter, and a position of the pupil.

### CITATION LIST

### Patent Literature

PTL 1: WO 2002/003853 A1

### SUMMARY

### Problem to be Solved

In order to measure the effectiveness of a medicine, it is necessary to perform blood concentration measurement or the like. On the other hand, since an appropriate dose of a medicine is strictly different for each individual, it is desirable that the subject himself/herself can easily measure the effectiveness of a medicine.

In view of the above-described problems, an object of the present disclosure is to provide an image processing device, an image processing method, and a storage medium capable of suitably generating information relating to an effectiveness of a medicine of a subject from an image obtained by photographing the subject.

### Means for Solving the Problem

One mode of the image processing device is an image processing device including:
a medication information acquisition means for acquiring medication information on a medicine used by a subject;
a photographing timing determination means for determining a photographing timing of the subject by a photographing means based on the medication information; and
a pupil information generation means for generating pupil information relating to a pupil of the subject based on an image generated by the photographing means at the photographing timing.

One mode of the image processing method is an image processing method executed by a computer, the image processing method including:
acquiring medication information on a medicine used by a subject;
determining a photographing timing of the subject by a photographing means based on the medication information; and
generating pupil information relating to a pupil of the subject based on an image generated by the photographing means at the photographing timing.

One mode of the storage medium is a storage medium storing a program executed by a computer, the program causing the computer to perform a process including:
acquiring medication information on a medicine used by a subject;
determining a photographing timing of the subject by a photographing means based on the medication information; and
generating pupil information relating to a pupil of the subject based on an image generated by the photographing means at the photographing timing.

### Effect

An example advantage according to the present invention is to suitably generate information relating to an effectiveness of a medicine of a subject from an image obtained by photographing the subject.

### BRIEF DESCRIPTION OF THE DRAWINGS

FIG. 1 illustrates a schematic configuration of a medication effect determination system according to a first example embodiment.

FIG. 2 illustrates a state at the time of determining a medication effect in a case where the medication effect determination system is one terminal device.

FIG. 3 illustrates an example of a hardware configuration of an image processing device common to each example embodiment.

FIG. 4 is an example of functional blocks of an image processing device relating to medication effect determination processing in the first example embodiment.

FIG. 5 is an example of a camera image obtained by photographing a face of a subject.

FIG. 6 is a graph showing an example of a change in a pupil size of a subject after the subject uses a certain medicine.

FIG. 7 is an example of a flowchart regarding medication effect determination processing executed by the image processing device.

FIG. 8 illustrates a schematic configuration of a medication effect determination system in a second example embodiment.

FIG. 9 is a block diagram of an image processing device in a third example embodiment.

FIG. 10 is an example of a flowchart executed by the image processing device in the third example embodiment.

### EXAMPLE EMBODIMENT

Hereinafter, example embodiments of an image processing device, an image processing method, and a storage medium will be described with reference to the drawings.

### First Example Embodiment

### (1) System Configuration

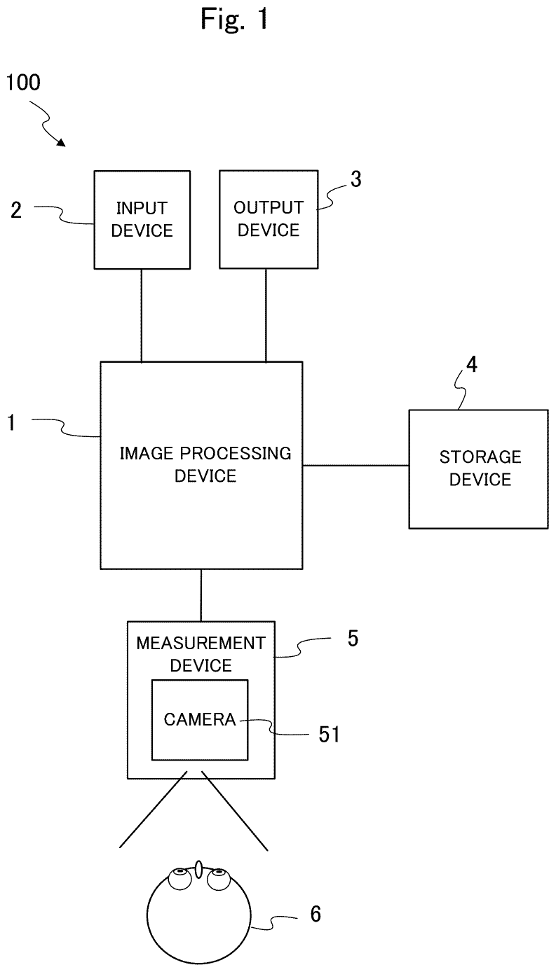

FIG. 1 illustrates a schematic configuration of a medication effect determination system 100 according to a first example embodiment. The medication effect determination system 100 is a system that simply determines the effectiveness (also referred to as “medication effect”) of a medicine used by a subject 6 based on an image obtained by photographing the face of the subject 6 with a visible light camera, and mainly includes an image processing device 1, an input device 2, an output device 3, a storage device 4, and a measurement device 5 including a camera (photographing device) 51. The medication effect determination system 100 may be used by the subject 6 for the purpose of self-care or may be used by the subject 6 under the instruction or request of a medical worker. The medication effect determination system 100 may be used, for example, for confirmation of a medication effect, adjustment of a medication amount and a medication interval, and the like. Hereinafter, the term “medication” is not limited to oral ingestion of a medicine by the subject 6, and includes a case where the subject 6 ingests a medicine through the skin.

The image processing device 1 measures the pupil of the subject 6 after the medication based on the face image (the same applies hereinafter, including a video which is a predetermined number of image sequences obtained in time series) of the subject 6 generated by the camera 51, and notifies the determination of the medication effect and information relating to the determination result of the medication effect based on the measurement result of the pupil. The image processing device 1 performs data communication with the input device 2, the output device 3, the storage device 4, and the measurement device 5 via a communication network or by wireless or wired direct communication.

The input device 2 is an interface that receives a user input (manual input). A user who inputs the information using the input device 2 may be the subject 6 himself/herself or a person who manages or supervises activities of the subject 6. The input device 2 may be, for example, various user input interfaces such as a touch panel, a button, a keyboard, a mouse, and an audio input device. The input device 2 supplies the input signal generated based on the user input to the image processing device 1.

The output device 3 outputs predetermined information based on the output signal supplied from the image processing device 1. In this case, the output signal includes at least one of a display signal and an audio signal. Then, the output device 3 displays information based on the display signal supplied from the image processing device 1, and outputs audio of the information based on the audio signal supplied from the image processing device 1. The output device 3 includes, for example, at least one of a display device such as a display or a projector and an audio output device such as a speaker.

The storage device 4 is a memory that stores various types of information necessary for the determination of the medication effect and the like. The storage device 4 may be an external storage device such as a hard disk connected to or incorporated in the image processing device 1, or may be a storage medium such as a flash memory. The storage device 4 may be a server device that performs data communication with the image processing device 1. The storage device 4 may include a plurality of devices.

The measurement device 5 is one or a plurality of sensors including the camera 51 which is a visible light camera. For example, the measurement device 5 may include an illuminance sensor for detecting a change in the amount of external light under the photographing environment of the camera image of the subject 6. The measurement device 5 supplies a signal measured by each sensor to the image processing device 1. Hereinafter, the image generated by the camera 51 is also referred to as a “camera image”. The camera 51 is an example of a “photographing means”.

The configuration of the medication effect determination system 100 illustrated in FIG. 1 is an example, and various changes may be made to the configuration. For example, the image processing device 1, the input device 2, the output device 3, the storage device 4, and the measurement device 5 may be achieved by one terminal device as a smartphone, a tablet terminal, or the like.

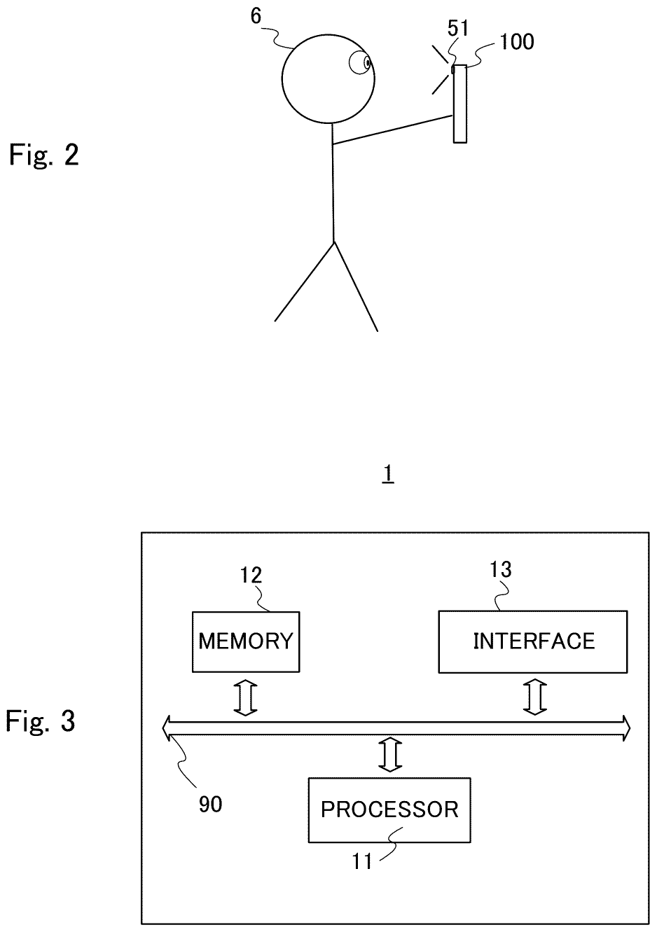

FIG. 2 illustrates a state at the time of photographing the camera image of the subject 6 in a case where the medication effect determination system 100 is one terminal device (for example, a smartphone). As illustrated in FIG. 2, the subject 6 holds the medication effect determination system 100, which is a terminal device, and adjusts the orientation of the terminal device such that the subject's face is included in the photographing range of the camera 51. The medication effect determination system 100 may be fixed to a tripod or the like. Then, in the state illustrated in FIG. 2, the subject 6 causes the medication effect determination system 100 to photograph a camera image of the subject 6 necessary for determining the medication effect in accordance with an instruction (guidance) output by the medication effect determination system 100.

### (2) Hardware Configuration

FIG. 3 illustrates the hardware configuration of the image processing device 1. The image processing device 1 includes, as hardware, a processor 11, a memory 12, and an interface 13. The processor 11, the memory 12, and the interface 13 are connected via a data bus 90.

The processor 11 functions as a controller (arithmetic device) that takes overall control of the image processing device 1 by executing a program stored in the memory 12. The processor 11 is, for example, a processor such as a central processing unit (CPU), a graphics processing unit (GPU), or a tensor processing unit (TPU). The processor 11 may include a plurality of processors. The processor 11 is an example of a computer.

The memory 12 includes various volatile memories and nonvolatile memories, such as a random access memory (RAM), a read only memory (ROM), and a flash memory. The memory 12 stores a program for executing processing to be performed by the image processing device 1. A part of the information stored in the memory 12 may be stored in one or more external storage devices capable of communicating with the image processing device 1, or may be stored in a storage medium detachable from the image processing device 1. The memory 12 may function as at least a part of the storage device 4.

The interface 13 is an interface for electrically connecting the image processing device 1 and another device. These interfaces may be a wireless interface such as a network adapter for wirelessly transmitting and receiving data to and from the another device, or may be a hardware interface for connecting to the another device by a cable or the like.

The hardware configuration of the image processing device 1 is not limited to the configuration illustrated in FIG. 2. For example, the image processing device 1 may include at least one of the input device 2, the output device 3, the storage device 4, and the measurement device 5.

### (3) Outline of Medication Effect Determination Processing

Next, medication effect determination processing, which is processing relating to determination of a medication effect, will be described. Schematically, the image processing device 1 determines the photographing timing of the camera image based on the medication information relating to the medication of the subject 6, and measures the pupil of the subject 6 based on the camera image photographed at the determined photographing timing. Then, the image processing device 1 notifies the determination of the medication effect and the information relating to the determination result based on a change in the measured pupil size of the subject 6 and the like. As a result, the image processing device 1 can accurately execute determination regarding the medication effect regardless of the type and amount of the medicine administered based on the camera image, and can notify the subject 6 or the like of a highly accurate determination result regarding the medication effect.

In general, the administration of a medicine causes a change in the pupil size according to the effectiveness of a medicine. For example, in a case where a medicine for the autonomic nervous system (for example, lexotan) is administered, mydriasis (dilatation) of the pupil associated with sympathetic nerve activity occurs. Pupil constriction occurs in a case where parasympathomimetics (for example, pilocarpine, phyzostigmine) are administered. In a case where parasympatholytics (for example, homatropine, tropicamide) are administered, mydriasis of the pupil occurs.

In consideration of the above, the image processing device 1 determines the medication effect based on the change in the pupil size. Since the timing at which the medication effect peaks differs depending on the medication content, the image processing device 1 determines the photographing timing of the camera image for specifying the change in the pupil size based on the medication information. As a result, the image processing device 1 determines the medication effect with high accuracy.

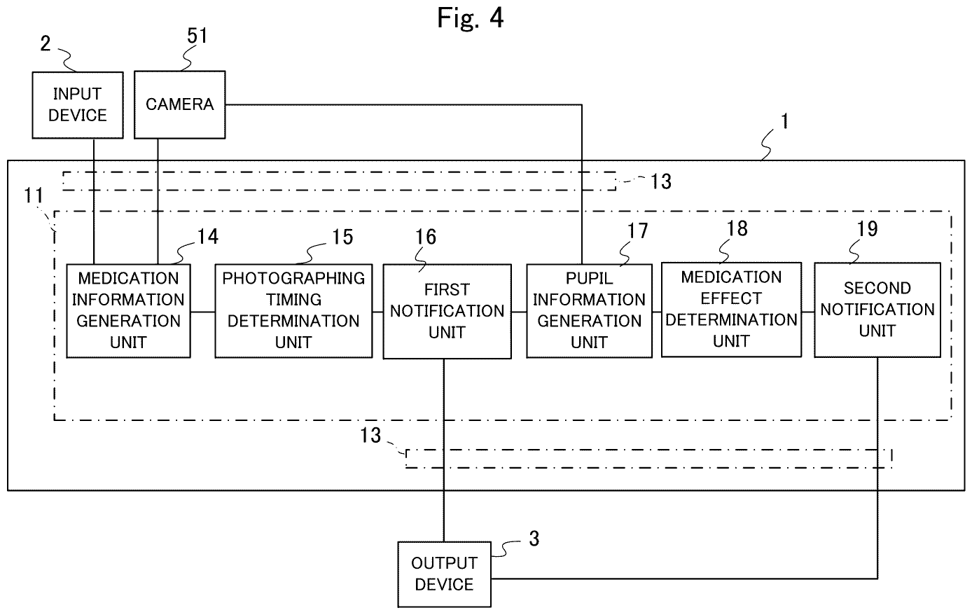

FIG. 4 is an example of functional blocks of the image processing device 1 relating to the medication effect determination processing. The processor 11 of the image processing device 1 functionally includes a medication information acquisition unit 14, a photographing timing determination unit 15, a first notification unit 16, a pupil information generation unit 17, a medication effect determination unit 18, and a second notification unit 19. In FIG. 4, blocks between which data is exchanged are connected by a solid line, but a combination of the blocks between which the data is exchanged is not limited to the illustrated combination. The same applies to diagrams of other functional blocks described later.

In a case where there is medication to the subject 6 (that is, use of medicine by the subject 6), the medication information acquisition unit 14 acquires medication information indicating at least medication contents (that is, the type and amount of medicine used) and medication time to the subject 6. Then, the medication information acquisition unit 14 supplies the acquired medication information to the photographing timing determination unit 15.

Here, a specific example of acquisition of the medication information will be described. In the first example, the medication information acquisition unit 14 generates the medication information based on the input of the subject 6 to the input device 2. For example, the medication information acquisition unit 14 causes the output device 3 to display an input screen for inputting the medication contents and the medication time, and acquires input information indicating the input on the input screen from the input device 2, thereby generating the medication information described above. In a case where the setting information of the medication content and the medication time is registered in advance in the storage device 4, the memory 12, or the like, the medication information acquisition unit 14 may generate the medication information based on the setting information.

In the second example, the medication information acquisition unit 14 acquires an image obtained by photographing a package (for example, a press through pack (PTP) sheet) packaging the medicine used by the subject 6, and analyzes the image by an arbitrary image recognition technology to recognize the medication content. Then, the medication information acquisition unit 14 recognizes the generation date and time indicated by the metadata or the like of the image as the medication time, and generates the medication information indicating the recognized medication content and medication time. In this example, the subject 6 or the administrator of the subject 6 photographs an image obtained by photographing a case (package) of the used medicine with the camera 51 or another camera after the subject 6 uses the medicine, and performs an input to designate the photographed image as an image to be analyzed by the medication information acquisition unit 14.

In the third example, in a case where an application for managing medication to the subject 6 is installed in the image processing device 1, the medication information acquisition unit 14 acquires medication information from the application.

The photographing timing determination unit 15 determines the photographing timing of the subject 6 based on the medication information supplied from the medication information acquisition unit 14, and supplies a photographing start instruction signal to the first notification unit 16 at the determined photographing timing. In this case, the photographing timing determination unit 15 determines each measurement timing of the pupil necessary for determining the medication effect as the photographing timing. The administration of the medicine causes a change in the pupil size according to the effectiveness of a medicine, and the effectiveness of a medicine varies depending on the type of medicine used and the like. In consideration of the above, the photographing timing determination unit 15 recognizes each measurement timing necessary for specifying the change in the pupil size based on the medication information, and determines each measurement timing as the photographing timing. Then, the photographing timing determination unit 15 supplies a photographing start instruction signal to the first notification unit 16 at the determined photographing timing. A method of determining the photographing timing by the photographing timing determination unit 15 will be described later.

In the case of receiving the photographing start instruction signal from the photographing timing determination unit 15, the first notification unit 16 notifies the subject 6 of the photographing timing by the output device 3. In this case, the first notification unit 16 supplies an output signal including at least one of an audio signal and a display signal to the output device 3 via the interface 13, thereby notifying the subject 6 of the photographing timing by the output device 3. As a result, the subject 6 adjusts the face of the subject 6 to be within the photographing range of the camera 51, and executes an operation to start photographing by the camera 51, and the like.

Preferably, in order to stabilize the focal point of the subject 6, the first notification unit 16 may instruct the subject 6 to turn the line of sight to a place a predetermined distance ahead (that is, to focus on). The above-described “predetermined distance” is, for example, a distance equal to or more than 6 m at which the focal length is substantially infinite. The place at the predetermined distance ahead is not limited to the place 6 m or more ahead, and may be any place where the distance to the subject 6 does not change.

In another suitable example, the first notification unit 16 may instruct the subject 6 such that the distance (photographing distance) between the subject 6 and the camera 51 becomes a predetermined distance based on the size of the iris of the subject 6 recognized from the camera image. In this case, the first notification unit 16 determines whether the size of the iris recognized from the camera image belongs to the appropriate size range. The above-described appropriate size range is a range of the size of the iris relevant to a range of the photographing distance within a distance range preferable for determining the medication effect, and is stored in advance in the storage device 4, the memory 12, or the like, for example. Then, in a case where the size of the recognized iris is out of the appropriate size range, the first notification unit 16 causes the output device 3 to output guidance information indicating that the photographing distance should be adjusted. In this case, the first notification unit 16 outputs guidance information (guidance) indicating that the photographing distance is shortened in a case where the size of the recognized iris is smaller than the appropriate size range, and outputs guidance information indicating that the photographing distance is lengthened in a case where the size of the recognized iris is larger than the appropriate size range. In this manner, the first notification unit 16 can suitably adjust the photographing distance to be a distance suitable for determining the medication effect.

In a case where the first notification unit 16 notifies the subject 6 of the photographing timing, the pupil information generation unit 17 generates information relating to the pupil of the subject 6 (also referred to as “pupil information”) based on the camera image obtained by photographing the subject 6. As a result, the pupil information generation unit 17 generates pupil information for each photographing timing determined by the photographing timing determination unit 15, and supplies the generated pupil information to the medication effect determination unit 18. The pupil information generation unit 17 calculates at least a pupil size (for example, a pupil diameter) of the subject 6 as the pupil information. In this case, the pupil information generation unit 17 may measure a predetermined number of pupil sizes based on a predetermined number of camera images obtained after the notification to the subject 6 by the first notification unit 16, and generate pupil information indicating the predetermined number of measured pupil sizes. In this case, the pupil information may be information indicating an average value, a maximum value, and other representative values of a predetermined number of pupil sizes.

The medication effect determination unit 18 determines the medication effect of the medicine used by the subject 6 based on the pupil information generated for each photographing timing. In this case, the medication effect determination unit 18 calculates one or a plurality of indexes (also referred to as a “pupil change index”) indicating a change relating to the pupil indicated by the pupil information. The pupil change index is, for example, a difference between the pupil size before the medication effect appears and the pupil size when the medication effect is at the peak, and is calculated as, for example, a difference between the pupil size based on the camera image generated at the photographing timing immediately after the medication and the pupil size at the photographing timing at the peak of the medication effect estimated based on the medication information. The above difference is relevant to the degree of miosis in a case where the pupil constricts, and is relevant to the degree of mydriasis in a case where the pupil is mydriatic. Then, the medication effect determination unit 18 determines the medication effect based on the calculated pupil change index, and supplies information relating to the determination result of the medication effect to the second notification unit 19.

Here, the determination of the medication effect by the medication effect determination unit 18 will be described.

For example, the medication effect determination unit 18 generates a determination result indicating whether the medication effect falls within an appropriate range. In a case where the medication effect is not within the appropriate range, the medication effect determination unit 18 may further generate a determination result indicating whether the medication effect is excessively large or excessively small.

In this case, for example, the medication effect determination unit 18 compares the calculated pupil change index with the reference value of the pupil change index, and determines the medication effect based on the comparison result. The reference value may be a standard value of the statistical pupil change index at the age of the subject 6 (that is, a statistical value of the pupil change index according to the attribute of the subject 6), or may be a past calculated value of the calculated value of the pupil change index of the subject 6. The reference value described above may be a threshold for determining whether the medication effect falls within an appropriate range. The medication effect determination unit 18 may use a model that performs inference regarding the medication effect and output information output by the model as information relating to the determination result of the medication effect. The above-described model is an equation, a lookup table, a machine learning model such as a neural network, or the like, and outputs an inference result regarding a medication effect in a case where a calculated value of various pupil change indexes (or a difference between the calculated value and a reference value, or the like) is used as an input, for example. Parameters and the like of the above-described model are stored in advance in the storage device 4, the memory 12, or the like. The medication effect determination unit 18 may output information for displaying a graph or the like indicating the calculated values of the various pupil change indexes and the relevant reference values in a comparable manner as information relating to the determination result of the medication effect.

The second notification unit 19 causes the output device 3 to display or output by voice the information relating to the determination result of the medication effect supplied from the medication effect determination unit 18. In this case, the second notification unit 19 generates an output signal including at least one of a display signal and an audio signal indicating information relating to the determination result of the medication effect, and supplies the output signal to the output device 3 via the interface 13. The second notification unit 19 may perform processing of notifying a third party other than the subject 6 of the information relating to the determination result of the medication effect, or may perform processing of storing the information relating to the determination result of the medication effect in the storage device 4, the memory 12, or the like.

The components of the medication information acquisition unit 14, the photographing timing determination unit 15, the first notification unit 16, the pupil information generation unit 17, the medication effect determination unit 18, and the second notification unit 19 described in FIG. 4 can be achieved, for example, by the processor 11 executing a program. Each component may also be achieved by recording a necessary program in an optional nonvolatile storage medium and installing the program as necessary. At least a part of these components is not limited to be achieved by software by a program, and may be achieved by a combination of any of hardware, firmware, and software, or the like. At least a part of these components may be achieved using, for example, a user-programmable integrated circuit such as a field-programmable gate array (FPGA) or a microcontroller. In this case, a program including the above components may be achieved by using the integrated circuit. At least a part of the components may include an application specific standard produce (ASSP), an application specific integrated circuit (ASIC), or a quantum processor (quantum computer control chip). In this manner, the components may be achieved by various types of hardware. The same applies to other example embodiments described later. These components may also be achieved by, for example, cooperation of a plurality of computers by using a cloud computing technology or the like.

### (4) Pupil Information

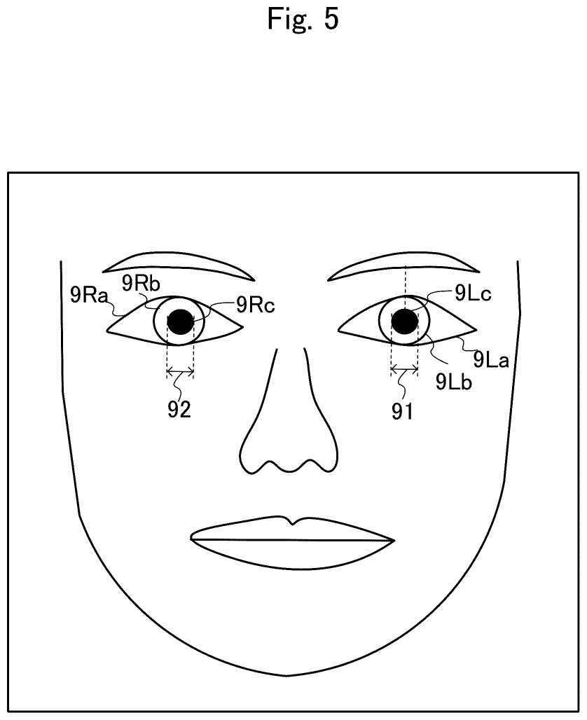

First, a specific example of the pupil information will be described. FIG. 5 is an example of a camera image obtained by photographing the face of the subject 6.

The image processing device 1 recognizes each region of the eyes 9Ra and 9La, the irises 9Rb and 9Lb, and the pupils 9Rc and 9Lc of the subject 6 based on an arbitrary image recognition technology from a camera image obtained by photographing the face of the subject 6. Then, the image processing device 1 calculates pupil information indicating at least the pupil size based on each recognized region.

For example, the image processing device 1 generates pupil information indicating a pupil size (that is, the pupil diameter) relevant to the lengths of the arrows 91 and 92. In a case where the pupil sizes of both eyes are calculated, the image processing device 1 may generate pupil information indicating an average of the calculated pupil sizes. In a case where only one eye of the subject 6 is displayed in the camera image, the image processing device 1 may generate pupil information indicating a pupil size of the displayed one eye.

Here, the image processing device 1 may perform normalization processing of converting the pupil size from a size based on the number of pixels on the camera image to a size of a predetermined scale (for example, an actual size), and generate pupil information indicating the normalized pupil size. In this case, for example, the image processing device 1 may normalize the pupil size based on the sizes of the irises 9Rb and 9Lb using characteristics that the sizes of the irises 9Rb and 9Lb have little individual differences and do not change overtime. In this case, for example, information indicating the relationship between the sizes of the irises 9Rb and 9Lb on the image and the sizes of the irises 9Rb and 9Lb after normalization is stored in advance in the storage device 4, the memory 12, or the like, and the image processing device 1 performs the above-described normalization with reference to this information. In another example, in a case where a plurality of cameras having different viewpoints are provided as the camera 51 in the medication effect determination system 100, the image processing device 1 may recognize the actual pupil size by three-dimensionally restoring the face of the subject 6 from the camera images of the plurality of cameras based on an arbitrary three-dimensional restoration technique such as SfM (Structure From Motion).

### (5) Photographing Timing

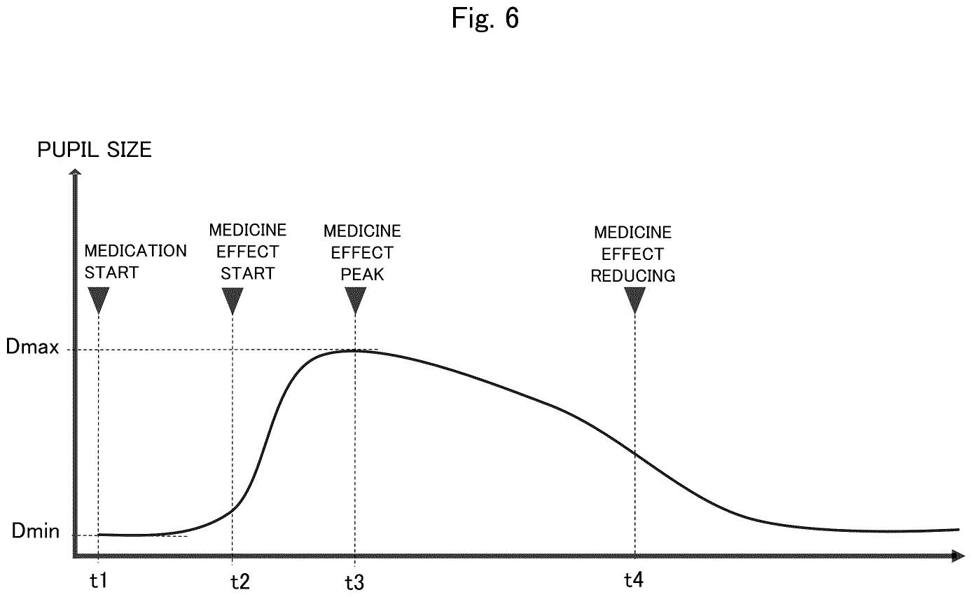

Next, the photographing timing determined by the photographing timing determination unit 15 will be described. FIG. 6 is a graph showing an example of a change in the pupil size of the subject 6 after the subject 6 uses a certain medicine. Here, as an example, a case where the subject 6 uses a medicine that causes mydriasis of the pupil by medication will be described.

The photographing timing determination unit 15 determines, as the photographing timing, at least a timing immediately after the start of the medication (time “t1” in FIG. 6 relevant to the medication time) and a timing at which the medication effect peaks (time “t3” in FIG. 6). Here, the pupil size at time t1 is relevant to the pupil size in a case where there is no medication effect, and is the minimum value “Dmin”. On the other hand, the pupil size at time t3 is relevant to the pupil size in a case where the pupil is maximally dilated due to the medication effect, and becomes the maximum value “Dmax”. In the case of a medicine that causes miosis, the pupil size becomes the minimum value at time t3.

Preferably, in addition to the above timings, the photographing timing determination unit 15 determines, as the photographing timing, at least one of a timing at which the medicine starts to work (time “t2” in FIG. 6) and a timing at which the medication effect is reduced (wom off) (time “t4” in FIG. 6).

Here, time t2 (that is, the timing at which the medicine starts to work), time t3 (that is, the timing at which the medication effect peaks), and time t4 (that is, the timing at which the medication effect is reduced) vary depending on the type (and amount) of medicine used by the subject 6. Therefore, the photographing timing determination unit 15 estimates these timings based on the medication content indicated by the medication information.

First, a method for estimating the timing at which the medication effect peaks will be described. Based on the medication content indicated by the medication information, the photographing timing determination unit 15 recognizes the time length from the medication time until the peak of the medication effect occurs (the time length from time t1 to time t3 in FIG. 6), and determines the time when the above-described time length has elapsed from the medication time as the timing at which the peak of the medication effect occurs. In this case, table information (that is, table information in which the above-described time length to be set for each assumed medication content is associated) indicating a correspondence relationship between the medication content and the above-described time length is stored in advance in the storage device 4, the memory 12, or the like. Then, the photographing timing determination unit 15 refers to the table information and determines the above-described time length from the medication content. In this case, the photographing timing determination unit 15 may determine the above-described time length from the type of medicine as the medication content, or may determine the above-described time length from both the type of medicine and the medication amount (usage amount).

Similarly, the photographing timing determination unit 15 refers to the table information indicating the correspondence relationship between the medication content and the time length from the medication time to the start of the effect of the medicine (the time length from time t1 to time t2 in FIG. 6), and estimates the timing of the start of the effect of the medicine from the medication content. The photographing timing determination unit 15 refers to table information indicating a correspondence relationship between the medication content and the time length from the medication time to the expiration of the medication effect (the time length from time t1 to time t4 in FIG. 6), and estimates the timing at which the medication effect expires from the medication content. The above-described table information is stored in advance in, for example, the storage device 4, the memory 12, or the like.

In this manner, the photographing timing determination unit 15 can accurately estimate each measurement timing of the pupil necessary for determining the medication effect based on the medication information, and can accurately determine the photographing timing.

Next, the calculation of the pupil change index will be supplementarily described with reference to FIG. 6. For example, the medication effect determination unit 18 calculates, as the pupil change index, a difference between the pupil size Dmin measured based on the camera image generated at time t1 immediately after the medication and the pupil size Dmax measured based on the camera image generated at time t3 at which the medication effect peaks. That is, the medication effect determination unit 18 calculates, as the pupil change index, a difference between the pupil size in a case where there is no medication effect and the pupil size when the pupil is maximally dilated due to the medication effect.

The photographing timing determination unit 15 may use, as the pupil size in a case where there is no medication effect, the pupil size based on the camera image obtained by photographing the subject 6 at any timing before the start of medication instead of using the pupil size at the timing immediately after the start of medication (time “t1” in FIG. 6). In this case, for example, a camera image obtained by photographing the subject 6 at an arbitrary timing before the start of medication is stored in the storage device 4, the memory 12, or the like, and the photographing timing determination unit 15 calculates the pupil size in a case where there is no medication effect based on the camera image. In the storage device 4, the memory 12, or the like, the pupil size of the subject 6 in a case where there is no medication effect may be stored in advance as the registration information.

The photographing timing determination unit 15 may determine a plurality of photographing timings in the vicinity of the estimated time t3 (that is, the timing at which the medication effect peaks), and estimate the maximum value “Dmax” of the pupil size (the minimum value in the case of a medicine in which the miosis occurs) by interpolation processing or another statistical method (for example, selection of a representative value such as the maximum value) based on the pupil size measured at each photographing timing. Similarly, the photographing timing determination unit 15 may determine a plurality of photographing timings for each of time t2 (that is, the timing at which the medicine starts to work) and time t4 (that is, the timing at which the medication effect is reduced), and determine the pupil size used to calculate the pupil change index based on the pupil size measured at each photographing timing.

### (6) Notification of Determination Result of Medication Effect

Next, a specific example of notification of the determination result of the medication effect by the second notification unit 19 will be described.

First, a case where a determination result indicating that the medication effect is excessively small is obtained (for example, in a case where the subject 6 uses a medicine that causes mydriasis but the degree of mydriasis is lower than the reference value) will be described.

In a first notification example in a case where it is determined that the medication effect is excessively small, the second notification unit 19 gives a notification for prompting confirmation as to whether the subject 6 has used the medicine according to the prescription in consideration of the possibility that the amount of medicine taken by the subject 6 has been small (that is, there is a possibility that the subject has taken a smaller amount than the prescribed amount). For example, the second notification unit 19 outputs guidance such as “Have you taken medicine as prescribed?” or “The medicine does not seem to work well, please consult a doctor at the next examination”.

In a second notification example in a case where it is determined that the medication effect is excessively small, the second notification unit 19 takes into consideration the possibility that the medicine is not effective, and notifies that the medicine is not effective due to the insufficient prescription amount for the subject 6 or that the medicine is not effective due to interactions with other substances. For example, the second notification unit 19 outputs guidance such as “Have you taken medicine as prescribed?”, “The medicine does not seem to work well, please consult a doctor at the next examination”, or “There is a possibility that you are taking food with bad interactions with the medicine. Please record what you ate today and consult a doctor or pharmacist at the next examination”.

In the second notification example described above, the second notification unit 19 may acquire information (also referred to as “intake information”) relating to a substance ingested (including oral and dermal ingestion) by the subject 6, and display the intake information and the pupil size calculated by the pupil information generation unit 17 in association with each other in time series. In this case, the intake information is, for example, information indicating at least the type (and amount) of the ingested substance and the intake time, and in a case where an application for managing the intake information of the subject 6 is installed in the image processing device 1, the second notification unit 19 acquires the intake information from the application. In another example, the second notification unit 19 acquires an image obtained by photographing a substance ingested by the subject 6 with the camera 51 or the like, and generates intake information by performing image analysis of the image using an arbitrary image recognition technology. In this case, the subject 6 makes an input to designate the photographed image as an image to be analyzed by the second notification unit 19.

In the second notification example described above, the second notification unit 19 may notify the subject 6 that interactions with other substances will occur in a case where the intake of a medicine indicated by the medication information (or a medicine registered as a medicine that the subject 6 regularly uses) and a substance that interacts poorly with the medicine are detected based on the intake information. In this case, for example, table information indicating a correspondence relationship between a medicine and a substance that interacts with the medicine is stored in the storage device 4, the memory 12, or the like, and the second notification unit 19 notifies the subject 6 that the interactions with other substances occurs in a case where the intake of the substance that causes interactions is detected based on the table information and the intake information.

Next, a case where a determination result indicating that the medication effect is excessive is obtained (for example, in a case where the subject 6 uses a medicine that causes mydriasis but the degree of mydriasis is higher than the reference value) will be described.

In the first notification example in a case where it is determined that the medication effect is excessive, in consideration of the possibility that the amount of medicine taken by the subject 6 has been large (that is, there is a possibility that the subject has taken an amount larger than the prescribed amount), a notification is given to prompt the subject 6 to confirm whether the medicine has been used according to the prescription. For example, the second notification unit 19 outputs guidance such as “Have you taken medicine as prescribed?” or “The medicine seems to be too effective, please consult a doctor at the next examination”.

In the second notification example in a case where it is determined that the medication effect is excessive, since the medicine is too effective, a notification suggesting a possibility that the prescription amount is large, a possibility that there is an influence of aging of the subject 6, a possibility that the type of medicine used is not suitable for the subject 6, or the like is performed. For example, the second notification unit 19 outputs guidance such as “Have you taken medicine as prescribed?”, “The medicine seems to be too effective, please consult a doctor at the next examination”, or “The medicine seems to be too effective. Aging and a large number of kinds of medicine may cause the medicine to be too effective. Record the medicine taken today and consult a doctor or pharmacist at the next examination”. In the above-described second notification example, the second notification unit 19 may acquire information relating to the medicine used by the subject 6, and display the acquired information together with the time-series measurement result of the pupil size measured by the pupil information generation unit 17 by the output device 3. The second notification unit 19 may store the information relating to the medicine used for the subject 6 and the time-series measurement result of the pupil size in the storage device 4, the memory 12, or the like in association with each other.

The subject 6 who has received the notification as described above can, for example, feed back the determination result of the medication effect to the doctor and consult the doctor so as to change his/her prescription amount. The subject 6 can determine the amount of medicine to be used according to the individual, reduce side effects, and make adjustments so as to most effectively utilize the medicine.

The second notification unit 19 may notify a third party other than the subject 6 of information relating to the determination result of the medication effect.

For example, the second notification unit 19 notifies a third party other than the subject 6 of information relating to the determination result of the medication effect in a case where the determination result that the medication effect on the subject 6 is not within the appropriate range continues a predetermined number of times. For example, in this case, contact information of a third party is stored in the storage device 4, the memory 12, or the like, and the second notification unit 19 transmits information relating to the determination result of the medication effect to the contact indicated by the contact information. Thus, the use of an appropriate type or amount of medicine by the subject 6 can be suitably promoted.

The second notification unit 19 may notify information relating to the determination result of the past medication effect of the subject 6 in addition to the determination result of the current medication effect of the subject 6 (that is, the latest determination result of the medication effect supplied from the medication effect determination unit 18). For example, the second notification unit 19 may display the determination result of the current medication effect of the subject 6 and the determination result of the past medication effect of the subject 6 in a comparable manner. The second notification unit 19 may notify the determination result of the individual medication effect obtained in the past, or may notify the statistical data in which the determination results of the individual medication effect obtained in the past are collected by statistical processing such as average processing. The second notification unit 19 may notify the determination result of the medication effect obtained a predetermined period ago (for example, 1 year ago) together with the determination result of the current medication effect of the subject 6. For example, every time the determination result of the medication effect is obtained, the second notification unit 19 stores the information relating to the determination result of the obtained medication effect in the storage device 4, the memory 12, or the like, so that the information relating to the past determination result of the medication effect is stored in the storage device 4, the memory 12, or the like.

Here, an effect of notification by the second notification unit 19 will be supplementarily described. For example, in a case where the subject 6 is an elderly person, by notifying the determination result of the current medication effect in comparison with the data of a predetermined period before (for example, 1 year before), the second notification unit 19 can cause the subject or a third party to suitably recognize the tendency in a case where the way the medicine works tends to change due to aging. In general, the functions of the liver and kidney decrease due to aging, it takes time to perform metabolism and excretion, and there is a possibility that a phenomenon that medicine is too effective occurs. In another example, in a case where the subject 6 has deteriorated in cognitive function or suffers from a mental disease, the second notification unit 19 can notify a third party of the determination result of the medication effect indicating the possibility that the intake amount has been too little or interactions with other substances has occurred. In this case, even if the subject is not aware of whether the intake amount is appropriate or effective, it is possible to give a sense of security to the third party or the subject or to encourage support by the third party for maintaining the medical condition of the subject 6 by notifying the third party. With respect to the subject 6 suffering from a mental disease, it is possible to suppress excessive intake or the like to reduce the frequency of hospitalization of the subject 6 or to suppress deterioration of the condition by the notification by the second notification unit 19.

### (5) Processing Flow

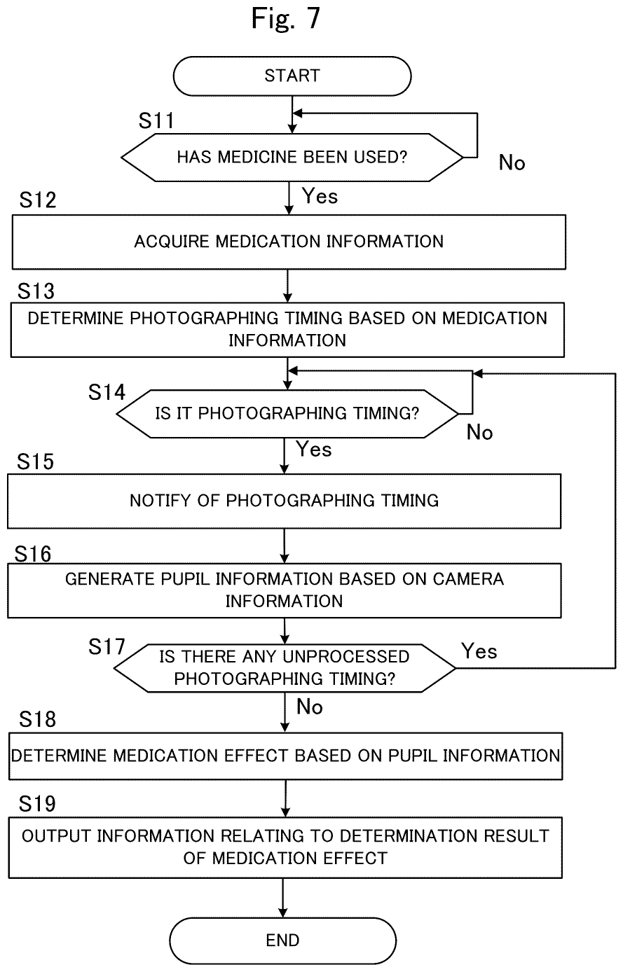

FIG. 7 is an example of a flowchart relating to medication effect determination processing executed by the image processing device 1.

First, the image processing device 1 determines whether a medicine has been used by the subject 6 (that is, medication) (step S11). Then, in a case where the image processing device 1 determines that a medicine has been used (step S11; Yes), medication information indicating the medication content and the medication time of the used medicine is acquired (step S12). For example, the image processing device 1 determines whether medicine is used in step S11 and acquires medication information in step S12 based on a user input (including registration of an image immediately after medication) by the input device 2. On the other hand, in a case where it is determined that the medicine has not been used (step S11; No), the image processing device 1 continuously determines whether a medicine is used in step S11.

After acquiring the medication information, the image processing device 1 determines the photographing timing based on the medication information (step S13). As a result, the image processing device 1 determines the timing immediately after the medication, the timing at which the medication effect estimated based on the medication information peaks, and the like as the photographing timing.

Then, the image processing device 1 determines whether any photographing timing determined in step S13 has come (step S14). Then, in a case where the image processing device 1 determines that any of the photographing timings determined in step S13 has come (step S14; Yes), the photographing timing is notified to the subject 6 by the output device 3 (step S15). As a result, the image processing device 1 prompts the subject 6 to perform an operation (including adjustment of a photographing distance) on the camera 51 necessary for pupil measurement. In this case, the image processing device 1 may output, by the output device 3, guidance for stabilizing the focal length, guidance for adjusting the photographing distance, and the like.

Then, the image processing device 1 generates pupil information indicating at least the pupil size of the subject 6 based on the camera image photographed by the camera 51 after the notification in step S15 (step S16). On the other hand, in a case where the image processing device 1 determines that it is not the photographing timing (step S14; No), the determination as to whether it is the photographing timing is continued in step S14.

Next, the image processing device 1 determines whether there is an unprocessed photographing timing (step S17). In other words, the image processing device 1 determines whether the processing of steps S15 and S16 has been completed for all the photographing timings determined in step S13. Then, in a case where there is an unprocessed photographing timing (step S17; Yes), the image processing device 1 returns to step S14.

On the other hand, in a case where there is no unprocessed photographing timing (step S17; No), the image processing device 1 determines the medication effect based on the pupil information generated in step S16 (step S18). Then, the image processing device 1 outputs information relating to the determination result of the medication effect (step S19). In this case, the image processing device 1 may display or output audio of the information relating to the determination result of the medication effect by the output device 3, may store the information in the storage device 4 or the like, or may transmit the information to a contact address of a third party other than the subject 6.

### Second Example Embodiment

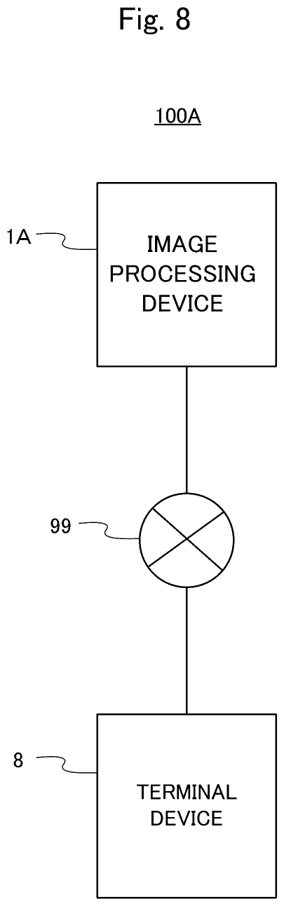

FIG. 8 illustrates a schematic configuration of a medication effect determination system 100A in a second example embodiment. The medication effect determination system 100A according to the second example embodiment includes an image processing device TA that functions as a server and a terminal device 8 that is used by a subject and functions as a client. The image processing device TA and the terminal device 8 perform data communication via a network 99. Hereinafter, the same components as those of the first example embodiment are appropriately denoted by the same reference signs, and description thereof will be omitted.

The terminal device 8 is a terminal used by a user as the subject, has an input function, a display function, a communication function, and a photographing function, and functions as the input device 2, the output device 3, the measurement device 5 including the camera 51, and the like illustrated in FIG. 1. The terminal device 8 may be, for example, a tablet terminal such as a personal computer or a smartphone, or a personal digital assistant (PDA). The terminal device 8 transmits the face image of the subject output by the camera 51 to the image processing device 1A via the network 99.

The image processing device 1A has the same hardware configuration as the hardware configuration of the image processing device 1 illustrated in FIG. 2, and the processor 11 of the image processing device 1A has the functional blocks illustrated in FIG. 4 described in the first example embodiment. Then, the image processing device 1A receives the camera image from the terminal device 8 via the network 99, and executes the medication effect determination processing for the subject. The image processing device 1A transmits an output signal for outputting a processing result to the terminal device 8 via the network 99 based on a display request from the terminal device 8.

As described above, the image processing device 1A in the second example embodiment performs the processing relating to the determination of the medication effect of the subject who is the user of the terminal device 8, and can suitably present the information relating to the determination result of the medication effect to the subject by the terminal device 8.

### Third Example Embodiment

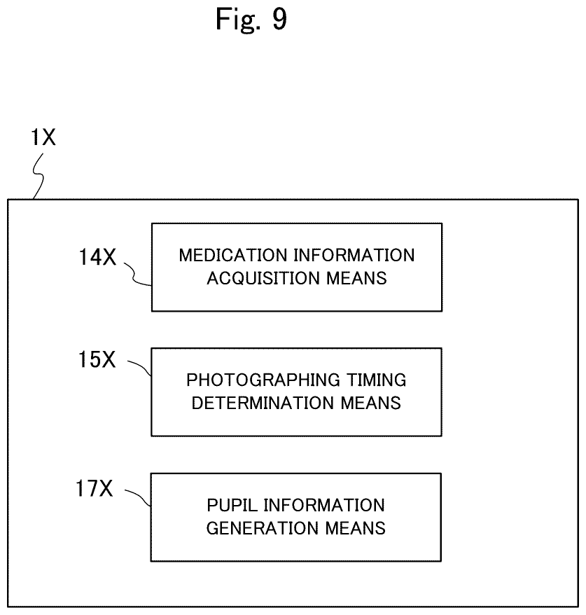

FIG. 9 is a block diagram of the image processing device 1X according to the third example embodiment. The image processing device 1X mainly includes a medication information acquisition means 14X, a photographing timing determination means 15X, and a pupil information generation means 17X. The image processing device 1X may be configured by plural devices.

The medication information acquisition means 14X is configured to acquire medication information on a medicine used by a subject. Examples of the medication information acquisition means 14X include the medication information acquisition unit 14 according to the first or second example embodiment.

The photographing timing determination means 15X is configured to determine a photographing timing of the subject by a photographing means based on the medication information. Examples of the photographing timing determination means 15X include the photographing timing determination means 15 according to the first or second example embodiment.

The pupil information generation means 17X is configured to generate pupil information relating to a pupil of the subject based on an image generated by the photographing means at the photographing timing. Examples of the pupil information generation means 17X include the pupil information generation unit 17 according to the first or second example embodiment.

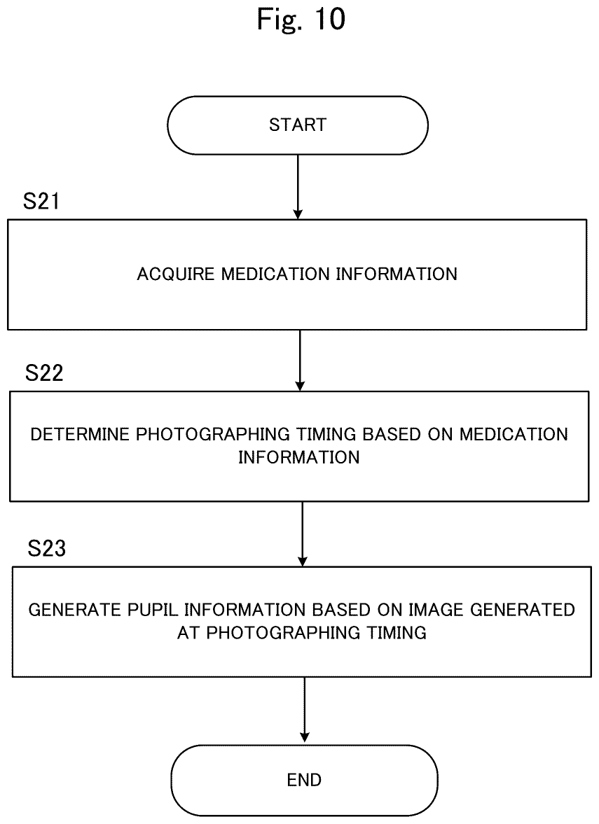

FIG. 10 illustrates a flowchart executed by the image processing device 1X according to the third example embodiment. The medication information acquisition means 14X acquires medication information on a medicine used by a subject (step S21). The photographing timing determination means 15X determines a photographing timing of the subject by a photographing means based on the medication information (step S22). The pupil information generation means 17X generates pupil information relating to a pupil of the subject based on an image generated by the photographing means at the photographing timing (step S23).

According to the third example embodiment, the image processing device 1X can suitably acquire pupil information, relating to a pupil of the subject, which changes in response to the use of the medicine by the subject.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments described above (including modifications, the same applies hereinafter) can be described as, but not limited to, the following Supplementary Notes.

### [Supplementary Note 1]

An image processing device comprising:
a medication information acquisition means for acquiring medication information on a medicine used by a subject;
a photographing timing determination means for determining a photographing timing of the subject by a photographing means based on the medication information; and
a pupil information generation means for generating pupil information relating to a pupil of the subject based on an image generated by the photographing means at the photographing timing.

### [Supplementary Note 2]

The image processing device according to Supplementary Note 1, further comprising a medication effect determination means for determining an effect of the medicine based on the pupil information.

### [Supplementary Note 3]

The image processing device according to Supplementary Note 2, wherein
the pupil information generation means generates the pupil information indicating at least a size of the pupil, and
the medication effect determination means makes a determination relating to the effect based on a change in a size of the pupil.

### [Supplementary Note 4]

The image processing device according to Supplementary Note 1, wherein the photographing timing determination means determines, as the photographing timing, at least a timing at which an effect of the medicine is estimated to peak based on the medication information.

### [Supplementary Note 5]

The image processing device according to Supplementary Note 1, wherein the photographing timing determination means determines, as the photographing timing, at least a timing at which it is estimated that an effect of the medicine starts to be effective based on the medication information.

### [Supplementary Note 6]

The image processing device according to Supplementary Note 1, wherein the photographing timing determination means determines, as the photographing timing, at least a timing at which an effect of the medicine is estimated to decrease based on the medication information.

### [Supplementary Note 7]

The image processing device according to Supplementary Note 1, further comprising a notification means for notifying the subject that the photographing timing is reached in a case where the photographing timing is reached.

### [Supplementary Note 8]

The image processing device according to Supplementary Note 7, wherein the notification means outputs guidance information regarding a distance between the subject and the photographing means based on a measurement result of an iris of the subject by the image.

### [Supplementary Note 9]

The image processing device according to Supplementary Note 2, further comprising a notification means for notifying a third party other than the subject of the determination result in a case where a determination result by the medication effect determination means indicates that the effect is not in an appropriate range continuously for equal to or more than a predetermined number of times.

### [Supplementary Note 10]

An image processing method executed by a computer, comprising:
acquiring medication information on a medicine used by a subject;
determining a photographing timing of the subject by a photographing means based on the medication information; and
generating pupil information relating to a pupil of the subject based on an image generated by the photographing means at the photographing timing.

### [Supplementary Note 11]

A storage medium storing a program for causing a computer to perform a process comprising:
acquiring medication information on a medicine used by a subject;
determining a photographing timing of the subject by a photographing means based on the medication information; and
generating pupil information relating to a pupil of the subject based on an image generated by the photographing means at the photographing timing.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All Patent and Non-Patent Literatures mentioned in this specification are incorporated by reference in its entirety.

DESCRIPTION OF REFERENCE NUMERALS

| | |
|---|---|
| 1, 1A, 1X | image processing device |
| 2 | Input device |
| 3 | Output device |
| 4 | Storage device |
| 5 | Measurement device |
| 8 | Terminal device |
| 11 | Processor |
| 12 | Memory |
| 13 | Interface |
| 51 | Camera |
| 90 | Data bus |
| 99 | Network |
| 100, 100A | Medication effect determination system |

What is claimed is:

1. An image processing device comprising:
at least one memory configured to store instructions; and
at least one processor configured to execute the instructions to:
acquire medication information on a medicine used by a subject;
determine a photographing timing of the subject based on the medication information; and
generate pupil information relating to a pupil of the subject based on an image generated by photographing the subject at the photographing timing.

2. The image processing device according to claim 1, wherein the at least one processor is configured to execute the instructions to determine an effect of the medicine based on the pupil information.

3. The image processing device according to claim 2, wherein
the at least one processor is configured to execute the instructions to
generate the pupil information indicating at least a size of the pupil, and
make a determination relating to the effect based on a change in a size of the pupil.

4. The image processing device according to claim 1, wherein the at least one processor is configured to execute the instructions to determine, as the photographing timing, at least a timing at which an effect of the medicine is estimated to peak based on the medication information.

5. The image processing device according to claim 1, wherein the at least one processor is configured to execute the instructions to determine, as the photographing timing, at least a timing at which it is estimated that an effect of the medicine starts to be effective based on the medication information.

6. The image processing device according to claim 1, wherein the at least one processor is configured to execute the instructions to determine, as the photographing timing, at least a timing at which an effect of the medicine is estimated to decrease based on the medication information.

7. The image processing device according to claim 1, wherein the at least one processor is configured to execute the instructions to notify the subject that the photographing timing is reached in a case where the photographing timing is reached.

8. The image processing device according to claim 7, wherein the at least one processor is configured to execute the instructions to output guidance information regarding a distance between the subject and the photographing means based on a measurement result of an iris of the subject by the image.

9. The image processing device according to claim 2, wherein the at least one processor is configured to execute the instructions to notify a third party other than the subject of the determination result in a case where a result of determining the effect by indicates that the effect is not in an appropriate range continuously for equal to or more than a predetermined number of times.

10. An image processing method executed by a computer, comprising:
acquiring medication information on a medicine used by a subject;
determining a photographing timing of the subject based on the medication information; and
generating pupil information relating to a pupil of the subject based on an image generated by photographing the subject at the photographing timing.

11. A non-transitory computer readable storage medium storing a program for causing a computer to perform a process comprising:
acquiring medication information on a medicine used by a subject;
determining a photographing timing of the subject based on the medication information; and
generating pupil information relating to a pupil of the subject based on an image generated by photographing the subject at the photographing timing.
